(19) United States
(12) Patent Application Publication
(10) Pub. No.: US 2003/0083730 A1
(43) Pub. Date: May 1, 2003

(54) Loading cartridge for self-expanding stent

(75) Inventor: Jonathan S. Stinson, Plymouth, MN (US)

Correspondence Address: Jay A. Stelacone, Esq., Finnegan, Henderson, Farabow,, Garrett & Dunner, L.L.P., 1300 I Street, N.W., Washington, DC, 20005-3315, US

(73) Assignee: Scimed Life Systems, Inc.

(21) Appl. No.: 09/983,728

(22) Filed: Oct. 25, 2001

(51) Int. Cl. A61F 2/06

(52) U.S. Cl. 623/1.11

(57) ABSTRACT

A device for loading a stent into a stent delivery system may comprise a tubular member, a funnel at least partially disposed in the tubular member, and a stent disposed in the tubular member. A distal portion of the funnel may separate a proximal portion of the stent from an inner surface of the tubular member.

### BACKGROUND OF THE INVENTION

1. Field of the Invention

The present invention relates to a system for loading a stent into a stent delivery device. More particularly, the present invention relates to a stent loading cartridge for loading a stent into a stent delivery catheter.

2. Description of Related Art

Stents are well-known endoprotheses. A conventional endoprosthetic stent includes a radially-expandable, tubular structure. After delivery to the region of a vessel being repaired or bridged, the tubular structure may be expanded radially from a compact delivery form to an expanded implantation form. Radial expansion of the stent affects implantation into the tissues of a vessel wall being repaired or bridged. The vessel can include, for example, a body canal, blood vessel, duct, other passage, and the like.

A conventional endoprosthetic stent can be mechanically expansive or self-expansive. A conventional mechanically-expansive stent initially possesses a radially compact form. The radially-compact stent may be loaded onto a delivery system, for example, a catheter, without further radial compression.

A conventional self-expansive stent initially possesses a radially-expanded form. Thus, the stent must be compressed radially as it is assembled onto a delivery system. Typically, an outer tubular structure retains the compressed stent until it is delivered to the region of a vessel being repaired or bridged. The stent is then released from its compressed state and self-expands to implant onto the vessel wall. While certain conventional metallic stents may be preloaded into a delivery system, for example, a delivery catheter, certain plastic bioabsorbable stents cannot be preloaded. If these plastic stents are preloaded, they may take a permanent set within the delivery catheter after a certain period of time, and they will not self-expand as desired.

Conventional stent delivery systems generally include a minimal transverse dimension so that a distal end of the delivery system can be navigated through and along a patient's lumens, or vessels, either in a percantaneous insertion procedure or through the working channel of an endoscope or laparoscope. Therefore, self-expanding stents must be radially compressed to at least that minimal transverse dimension in order to be loaded into the delivery system. This may be conventionally accomplished by manually squeezing one end of the stent to reduce its diameter and inserting the radially-compressed stent into the distal end of a delivery catheter or, alternatively, into a funnel disposed at the distal end of a delivery catheter.

For example, referring to FIG. 6, a loading funnel 158 may be removably attached to a distal end of the delivery catheter 110. The distal end 144 of an outer member 140 is slidably retracted away from the distal end 124 of an inner member 120 in the axial direction of the catheter. A physician causes relative movement between the inner member and the outer member with loading funnel by holding the inner member at, for example, the distal end or proximal end, and slidably moving the outer member relative to the inner member in an axial direction away from the distal end of the inner member.

As the outer member is retracted, a holding sleeve for the radially compressed stent 126 adhered about the inner member is exposed. A physician or other user passes the stent 190 over the tip 136 of the distal end of the inner member and onto the holding sleeve. To do so, the user gently compresses the stent in a radially direction and fits it into the loading funnel until a proximal end of the stent reaches a desired position, as shown in FIG. 6. While holding the stent stationary in a radially-compressed configuration, the loading funnel and outer member are advanced toward the distal end of the inner member. Again, the relative movement between the inner member and the outer member with loading funnel is effectuated by holding the inner member at, for example, the distal end or proximal end and slidably moving the outer member relative to the inner member in an axial direction toward the distal end of the inner member. The outer member is advanced until the stent is fully constrained between the inner member and outer member and between the holding sleeve and outer member. The holding sleeve fills any gap between the radially compressed stent and the inner member so as to prevent the stent from slipping in an axial direction during loading and deployment. The friction between the holding sleeve and the stent prevents stent movement. The length of the holding sleeve need not be as long as the radially compressed stent in order to be effective.

As a result, conventional loading of a stent into a delivery system may require a high level of manual dexterity and significant practice by a user, for example, a physician, nurse, or the like. Since many users will not have significant experience with loading stents, the loading process may be difficult. Further, the stents may be structurally damaged by mis-handling, and the sterility of the stents may be compromised by contamination through over-handling. This ineffective and inefficient loading may prolong a surgical procedure thereby increasing the trauma and risk to the patient as well as increasing costs.

### SUMMARY OF THE INVENTION

As embodied and broadly described herein, there is provided a loading cartridge for a self-expanding stent delivery system. A loading cartridge for a stent delivery system may comprise a tubular member, an unconstrained stent holding sleeve associated with an inner surface of the tubular member, a funnel at least partially disposed in the tubular member, and a stent spaced from and disposed in the tubular member.

Another optional aspect of the invention provides a method of loading a stent onto a stent delivery system. A method of loading a stent onto a stent delivery system may comprise connecting a loading cartridge to a catheter, where the loading cartridge comprises a stent in a radially-expanded configuration, and funneling the stent onto the catheter.

According to another optional aspect, a device for loading a stent into a stent delivery system may comprise a tubular member, a funnel at least partially disposed in the tubular member, and a stent disposed in the tubular member. A distal portion of the funnel may separate a proximal portion of the stent from an inner surface of the tubular member.

Yet another optional aspect provides a method of loading a stent onto a stent delivery system. The method may comprise providing a stent within a tubular member. A distal end of a funnel may separate at least a portion of the stent from the tubular member. The method may further comprise attaching a proximal end of the funnel to a catheter, and moving the stent through the funnel and onto the catheter.

According to still another optional aspect, a stent delivery system may comprise a catheter, a funnel, a tubular member, and a stent. A proximal end of the funnel may be attached at a distal end of the catheter. The funnel may be at least partially disposed in the tubular member, and the stent may be disposed in the tubular member. A distal portion of the funnel may separate a proximal portion of the stent from an inner surface of the tubular member.

Aside from the structural and procedural arrangement set forth herein, there could be a number of other arrangements. It is to be understood that both the foregoing description and the following description are exemplary.

### BRIEF DESCRIPTION OF THE DRAWINGS

The accompanying drawings, which are incorporated in and constitute part of the specification, illustrate a presently preferred embodiment of the invention and, together with the general description given above and detailed description of the preferred embodiments given below, serve to explain the principles of the invention.

FIG. 1 is a plan view of an exemplary stent delivery system;

FIG. 2 is a plan view of the stent delivery system of FIG. 1 including an exemplary loading cartridge according to the invention;

FIG. 3 is a partial, cross-sectional view of the system of FIG. 2 while in an exemplary state of stent loading according to the invention; and

FIG. 4 is a partial, cross-sectional view of the system of FIG. 2 while in another exemplary state of stent loading according to the invention;

FIG. 5 is a partial, cross-sectional view of the system of FIG. 2 while in another exemplary state of stent loading according to the invention; and

FIG. 6 is a partial, cross-sectional view of a stent delivery system in a state of conventional stent loading.

### DETAILED DESCRIPTION

Reference now will be made in detail to the present preferred embodiments of the invention, examples of which are illustrated in the accompanying drawings, in which like numerals designate like elements.

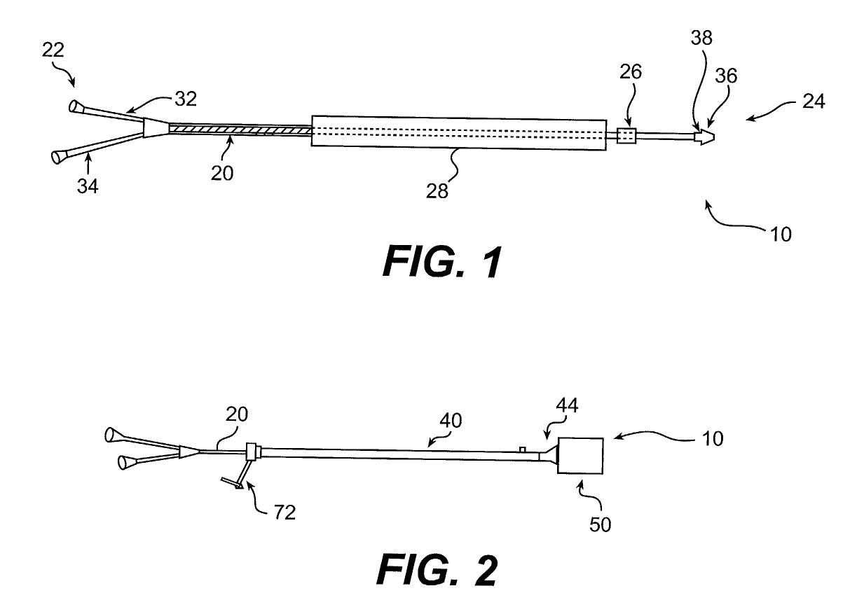

In accordance with the present invention, there is provided a delivery system for a self-expanding stent. As embodied herein and shown in FIGS. 1-2, the delivery system 10, for example, a catheter, may be configured to deploy a self-expanding stent. The stent may, for example, repair or bridge a damaged vessel of a patient's body. The catheter 10 may include an inner member 20 and an outer member 40. Optionally, the inner and outer members 20, 40 are tubular-shaped. In one exemplary embodiment, a portion of the inner member 20 may be formed of stainless steel. However, the invention in its broadest sense is not limited by the shape, size, composition, or type of the inner member 20.

FIG. 1 illustrates the delivery system with the outer member 40 removed. In FIG. 1, the inner member 20 has a proximal end 22 and a distal end 24. Spaced from the distal end 24, the inner member 20 may include a holding sleeve 26 for the constrained stent. In one exemplary embodiment, the holding sleeve 26 may be coaxially mounted about the inner member 20 and sized and configured such that a self-expanding stent can be placed around it. The holding sleeve 26 may retain the positioning of the stent during delivery and re-constrain procedures by cooperating with the outer member 40 to prevent axial movement of the stent. Optionally, the inner member 20 may also be provided with an inflatable device (not shown) positioned between the holding sleeve 26 and the distal end 24. An example of such an optional embodiment is described in detail in co-pending U.S. patent application Ser. No. 09/753,448.

As shown in FIG. 1, the inner member 20 may include a first lumen tube 32 and/or a second lumen tube 34 configured to receive a medical guidewire (not shown) and/or provide a fluid passage through the inner member 20. The first and second lumen tubes 32, 34 may be arranged concentrically or side-by-side. Alternatively, the inner member 20 may include a single lumen tube or any other configuration known in the art.

In an exemplary embodiment of the invention, the distal end 24 of the inner member 20 includes a tapered tip 36. The tapered tip 36 may provide easier delivery and maneuverability, for example, when using the delivery system in combination with a medical guidewire. In addition, the tapered tip 36 may include a surface 38 extending radially outward from the inner member 20 and forming a seat against which the outer member 40 can rest.

FIG. 2 illustrates the delivery system with the outer member 40 coaxially positioned about the inner member 20. The outer member 40 may be slidably mounted about the inner member 20 to permit relative axial movement between them. As shown in FIG. 2, a loading cartridge 50 may be removably attached to a distal end 44 of the outer member 40 for loading a stent onto the catheter 10.

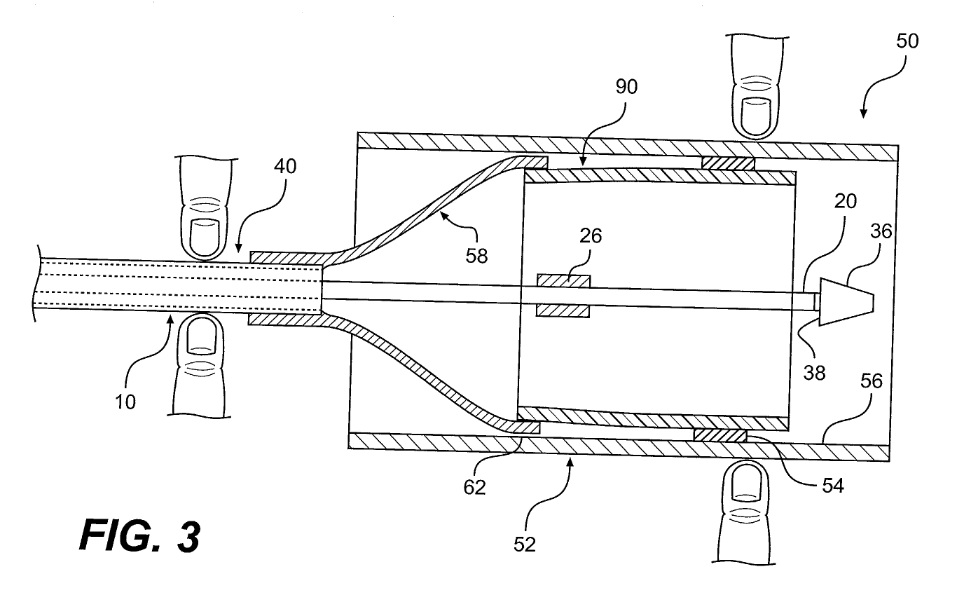

FIG. 3 illustrates the loading cartridge 50 in combination with the catheter 10. The loading cartridge 50 may comprise an tubular member 52 and a holding sleeve 54 for the unconstrained stent disposed at an inner surface 56 of the tubular member 52. The loading cartridge 50 may also include a funnel 58 sized and shaped to assist with radial compression of a self-expanding stent as the stent is loaded onto the delivery system. The loading cartridge 50 may further comprise a self-expanding stent 90 disposed in the tubular member 52. The stent 90 may be made, for example, of bioabsorbable poly-1-lactide filaments braided in a tubular mesh configuration. However, the invention in its broadest sense is not limited by the shape, size, composition, or type of the self-expanding stent 90.

The loading cartridge 50, in an optional embodiment, may have the tubular member 52, holding sleeve 54, funnel 58, and stent 90 assembled in the fashion shown in FIG. 3 and available to a practitioner in this pre-assembled fashion. Then, in use, the practitioner may position a portion of the funnel 58 at a distal end of the catheter 10 for loading the stent 90 onto the catheter 10.

As shown in FIG. 3, the self-expanding stent 90 may be disposed in the tubular member 52 in an uncompressed position. A portion of the stent 90 may be disposed in a distal end 62 of the funnel 58. Optionally, at least a portion of the funnel 58 may comprise a material having a low coefficient of friction, for example, TEFLON, or a non-toxic lubricant. The stent 90 may also extend through the holding sleeve 54 disposed in the tubular member 52. Optionally, at least a portion of the holding sleeve 54 may comprise a material having a high coefficient of friction, for example, cured silicone. In an optional embodiment, the holding sleeve 54 may be fixedly mounted to the inner surface of the tubular member 52, for example, by an adhesive.

It should be appreciated that the holding sleeve 54 may be eliminated from loading cartridge 50. Alternatively, the loading cartridge 50 may be configured in a such manner that a portion of the tubular member 52 or another alternate structure may restrain the stent 90 from moving axially in the loading cartridge.

Referring to FIG. 3, the loading cartridge 50 may be positioned on a distal end 44 of the outer member 40 of the catheter 10 for loading the stent 90. The funnel 58 may have an edge on it to line up with the distal edge of the outer tube of the catheter 10. Optionally, the loading cartridge 50 may engage the outer member 40 in a friction fit relationship. Alternatively, the loading cartridge 50 may be attached to the outer member 40 by other well known methods, for example, screw-fastening. The outer member 40 may be moved relative to the inner member 20 in a direction away from the tip 36. As a result, the holding sleeve 26 on the inner member 20 may be uncovered by the outer member 40.

The stent 90 may be further moved into the funnel 58 by moving the outer member 40, relative to the inner member 20, in a direction toward the tip 36. The funnel 58 may move substantially with the outer member 40 in the direction toward the tip 36. At least a portion of the outer surface of the funnel 58 may comprise a material with a low coefficient of friction to facilitate movement relative to the tubular member 52. The tubular member 52 may comprise, for example, a polymer such as polyethylene or polyurethane. The outer member 40 and the funnel 58 may move towards the tip 36 until the funnel 58 contacts the holding sleeve 54.

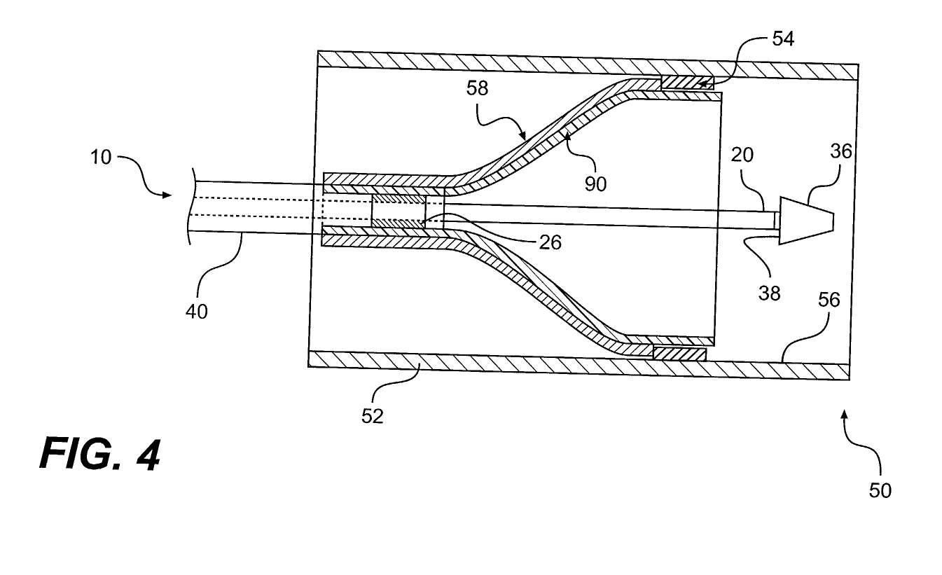

Referring to FIG. 4, an interior diameter of the funnel 58 may be less than the inner diameter of the holding sleeve 54. Thus, as the funnel 58 nears the holding sleeve 54, the stent 90 may be radially compressed from its original configuration in association with the holding sleeve 54. Optionally, the stent 90 may no longer contact the holding sleeve 54, even though the stent 90 may still extend through the holding sleeve 54. As a result, the tubular member 52 and holding sleeve 54 may be separated from the funnel 58 and the stent 90 with little or no frictional resistance, for example, by sliding in a direction away from the catheter 10.

In one exemplary embodiment, the holding sleeve 26 on the inner member 20 of the catheter 10 may contact the stent 90 at some point in time prior to the funnel 58 engaging the holding sleeve 54 associated with the loading cartridge 50. The holding sleeve 26 may axially restrain movement of the stent 90 by cooperating with the outer member 40 of the catheter 10. Alternatively, the holding sleeve 26 may be brought into contact with the stent 90 after removing the tubular member 52 and further moving the outer member 40 of the catheter 10 towards the tip 36.

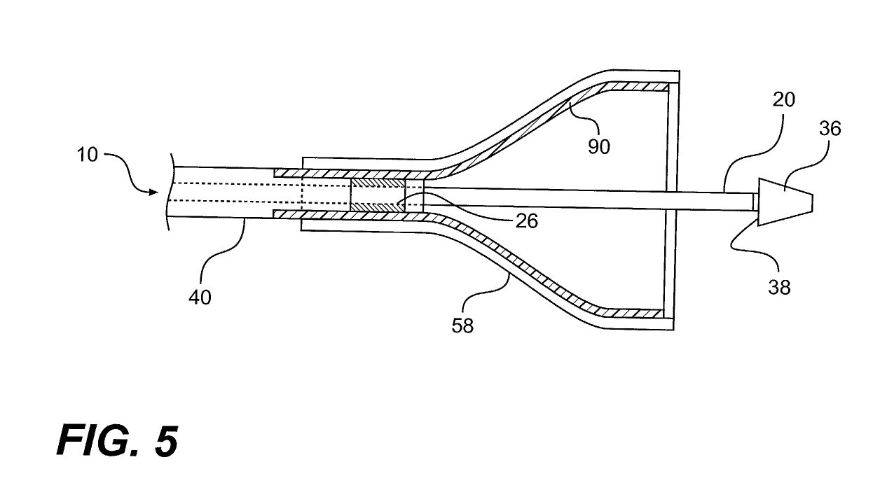

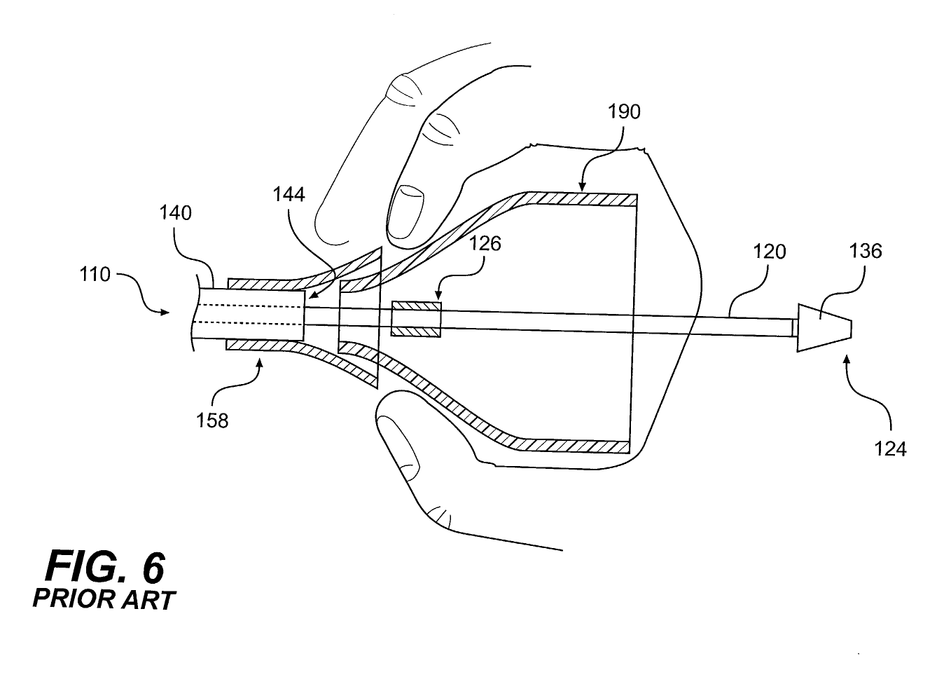

Referring to FIG. 5, the stent 90 may be radially compressed along its entire length by continuing movement of the outer member 40, relative to the inner member 20, towards the tip 36. In one optional embodiment, the outer member 40 may be moved until its distal end 44 contacts the surface 38 of the tip 36. Alternatively, if the stent 90 does not extend to the surface 38, movement of the outer member 40 may be stopped short of the surface 38 of the tip 36.

Once the self-expanding stent 90 is loaded onto the catheter, the user delivers the delivery system along a medical guidewire or through an endoscope or laparoscope to the area of the vessel to be repaired or bridged. Once delivered to the appropriate location, the stent is released and allowed to self-expand, thereby implanting itself onto the vessel wall. The outer member 40 may release the self-expanding stent 90 to a radially-expanded position as the outer member 40 slides relative to the inner member 20 in a direction away from the surface 38.

In an optional embodiment, the delivery system may include a spacing jacket 28 coaxially positioned about the inner member 20 and inside the outer member 40. The spacing jacket 28 may reduce snaking, coiling, or twisting of the inner member within the outer member, particularly during delivery through a tortuous anatomy.

In another optional embodiment, the delivery system may include a fluid port 72. The fluid port 72 may be a conduit having a stopcock for connecting a syringe or any other device known in the art. The fluid may be used, for example, to flush the region between the inner member 20 and outer member 40.

It should be appreciated that a loading cartridge may be attached to a catheter during the manufacturing and assembly process. For example, the loading cartridge may be attached to the catheter in a friction fit relationship during manufacturing and assembly. After loading the stent at or near a time and point of use, the loading cartridge may be removed by sliding the funnel off of the catheter. Optionally, the funnel may include a removable strip along its length, wherein removal of the strip may relax the interference fit and facilitate removal of the funnel. Alternatively, the catheter and loading cartridge may be assembled and distributed separately and attached to one another at or near the time and point of use by a practitioner.

It will be apparent to those skilled in the art that various modifications and variations can be made to the apparatus and method described herein. Other embodiments of the invention will be apparent to those skilled in the art. It is intended that the specification and examples be considered as exemplary only.

We claim:

1. A loading cartridge for a stent delivery system, the loading cartridge comprising:
a tubular member;
a holding sleeve associated with an inner surface of the tubular member;
a funnel at least partially disposed in the tubular member; and
a stent spaced from and disposed in the tubular member.

2. The loading cartridge of claim 1, wherein the stent is associated with at least one of the holding sleeve and the funnel.

3. The loading cartridge of claim 2, wherein the stent is associated with an inner surface of the holding sleeve.

4. The loading cartridge of claim 2, wherein the stent is associated with an inner surface of the funnel.

5. The loading cartridge of claim 1, wherein the stent is axially restrained by the holding sleeve.

6. The loading cartridge of claim 1, wherein the funnel is configured to connect with a catheter.

7. The loading cartridge of claim 1, wherein the stent comprises a self-expanding stent.

8. The loading cartridge of claim 1, wherein the stent comprises a radially-expanded position while disposed in the tubular member.

9. The loading cartridge of claim 1, wherein the funnel comprises a maximum inside diameter in the tubular member and a minimum inside diameter outside of the tubular member.

10. The loading cartridge of claim 9, wherein the maximum inside diameter of the funnel is less than an inside diameter of the holding sleeve.

11. A method of loading a stent onto a stent delivery system, comprising:
connecting a loading cartridge to a catheter, the loading cartridge containing a stent in a radially-expanded configuration; and
funneling the stent onto the catheter.

12. The method of claim 11, wherein said funneling the stent comprises:
moving an outer member of the catheter towards the stent; and
radially compressing the stent with a funnel.

13. The method of claim 12, wherein said radially compressing comprises radially compressing the stent to a diameter configured to enter the outer member of the catheter.

14. The method of claim 13, further comprising:
removing an outer member of the loading cartridge from the funnel and the stent.

15. The method of claim 14, further comprising moving the outer member of the catheter, after said removing, until the stent is radially-compressed and covered by the outer member of the catheter along its entire length.

16. The method of claim 15, further comprising removing the funnel from the stent.

17. The method of claim 12, wherein said connecting comprises connecting the funnel to the outer member of the catheter.

18. The method of claim 11, further comprising restraining axial movement of the stent relative to the cartridge.

19. The method of claim 11, further comprising contacting the stent with a holding sleeve associated with the catheter.

20. The method of claim 19, further comprising restraining axial movement of the stent relative to the holding sleeve.

21. A device for loading a stent into a stent delivery system, comprising:
a tubular member;
a funnel at least partially disposed in the tubular member; and
a stent disposed in the tubular member, wherein a distal portion of the funnel separates a proximal portion of the stent from an inner surface of the tubular member.

22. The device of claim 21, wherein the stent is axially restrained in the tubular member.

23. The device of claim 21, further comprising a holding sleeve within the tubular member, the holding sleeve separating the stent from the inner surface of the tubular member.

24. The device of claim 23, wherein the stent is axially restrained by the holding sleeve.

25. The device of claim 21, wherein the funnel is configured to connect with a catheter.

26. The device of claim 21, wherein the stent comprises a self-expanding stent.

27. The device of claim 21, wherein the stent comprises a radially-expanded position while disposed in the tubular member.

28. The device of claim 23, wherein the funnel comprises a maximum inside diameter in the tubular member and a minimum inside diameter outside of the tubular member.

29. The device of claim 28, wherein the maximum inside diameter of the funnel is less than an inside diameter of the holding sleeve.

30. A method of loading a stent onto a stent delivery system, comprising:
providing a stent within a tubular member, wherein a distal end of a funnel separates at least a portion of the stent from the tubular member;
attaching a proximal end of the funnel to a catheter; and
moving the stent through the funnel and onto the catheter.

31. The method of claim 30, wherein said providing a stent comprises providing the stent in a radially-expanded configuration.

32. The method of claim 30, wherein said moving the stent comprises:
moving an outer member of the catheter towards the stent; and
radially compressing the stent with the funnel.

33. The method of claim 32, wherein said radially compressing comprises radially compressing the stent to a diameter configured to enter the outer member of the catheter.

34. The method of claim 33, further comprising:
removing an outer member of the tubular member from the funnel and the stent.

35. The method of claim 34, further comprising moving the outer member of the catheter, after said removing, until the stent is radially-compressed and covered by the outer member of the catheter along its entire length.

36. The method of claim 35, further comprising removing the funnel from the stent.

37. The method of claim 32, wherein said attaching comprises attaching the funnel to the outer member of the catheter.

38. The method of claim 30, further comprising restraining axial movement of the stent relative to the tubular member.

39. The method of claim 30, wherein said moving the stent comprises contacting the stent with a holding sleeve associated with the catheter.

40. The method of claim 39, further comprising restraining axial movement of the stent relative to the holding sleeve.

41. A stent delivery system, comprising:
a catheter;
a funnel, a proximal end of the funnel being attached at a distal end of the catheter;
a tubular member, the funnel being at least partially disposed in the tubular member; and
a stent disposed in the tubular member, wherein a distal portion of the funnel separates a proximal portion of the stent from an inner surface of the tubular member.

42. The system of claim 41, wherein the catheter comprises an outer member, the funnel being attached to a distal end of the outer member.

43. The system of claim 41, wherein the stent is axially restrained in the tubular member.

44. The system of claim 41, further comprising a holding sleeve within the tubular member, the holding sleeve separating the stent from the inner surface of the tubular member.

45. The system of claim 44, wherein the stent is axially restrained by the holding sleeve.

46. The system of claim 41, wherein the funnel is configured to connect with a catheter.

47. The system of claim 41, wherein the stent comprises a self-expanding stent.

48. The system of claim 41, wherein the stent comprises a radially-expanded position while disposed in the tubular member.

49. The system of claim 44, wherein the funnel comprises a maximum inside diameter in the tubular member and a minimum inside diameter outside of the tubular member.

50. The system of claim 49, wherein the maximum inside diameter of the funnel is less than an inside diameter of the holding sleeve.
